(19) United States
(12) Patent Application Publication
(10) Pub. No.: US 2025/0010014 A1
(43) Pub. Date: Jan. 9, 2025

(54) COMPONENT ADDITIONS FOR AN AUTOMATIC RESCUE BREATHING UNIT

(71) Applicant: Daniel A. McCarthy, Tyler, TX (US)

(72) Inventor: Daniel A. McCarthy, Tyler, TX (US)

(21) Appl. No.: 18/763,949

(22) Filed: Jul. 3, 2024

Related U.S. Application Data
(60) Provisional application No. 63/520,678, filed on Aug. 21, 2023.
(60) Provisional application No. 63/512,128, filed on Jul. 6, 2023.

(51) Int. Cl. A61M 16/10; A61M 16/00; A61M 16/06

(52) U.S. Cl. CPC A61M 16/1005; A61M 16/0003; A61M 16/022; A61M 16/0605

(57) ABSTRACT

A rescue breathing apparatus and various components for use with the rescue breath apparatus are disclosed. The apparatus is an automatic rescue breathing unit (ARBU) device that includes at least two sub-systems—an airway device and a ventilation unit. In some instances, a compression pad is implemented with the ARBU device to prevent breaths during chest compressions. A breath pause button may be added to the ARBU device to pause breaths as controlled by personnel operating the ARBU device (e.g., a practitioner). A rescue breath kit with individual ARBU devices for different types of patients is also disclosed.

### PRIORITY CLAIM

This application claims the benefit of priority to U.S. Provisional Application No. 63/512,128 filed Jul. 6, 2023 and to U.S. Provisional Application No. 63/520,678 filed Aug. 21, 2023, both of which are incorporated herein by reference.

### RELATED APPLICATION

This application is related to U.S. patent application Ser. No. 18/525,346 filed Nov. 30, 2023, the disclosure of which is incorporated herein by reference.

### BACKGROUND

### 1. Field of the Invention

The disclosed embodiments generally relate to an apparatus for providing air/oxygen to a subject, and more particularly to components that can be added for improving operation of the apparatus.

### 2. Description of Related Art

Medical emergencies often call on one or more people to provide life-saving support. For example, CPR may be performed when a person is not breathing, or breathing inadequately (e.g., during cardiac arrest). CPR generally involves providing air into a person's lungs via the mouth, or mouth and nose, and/or performing a series of chest compressions. This may be performed repeatedly to help oxygenate and circulate the blood. Blowing air into the victim's mouth forces air into the lungs to replace spontaneous respiration and compressing the chest compresses the heart to maintain blood circulation. In a situation in which the heart has stopped beating, performing CPR is intended to maintain a flow of oxygenated blood to the brain and heart, thereby delaying tissue death and extending the opportunity for a successful resuscitation without permanent brain damage. Defibrillation and other advanced life support techniques may also be used to improve the outcome for a victim of cardiac arrest.

CPR techniques can vary depending on the person needing assistance. For example, administering CPR to an adult generally includes providing a set number of full breaths via the mouth, whereas administering CPR to an infant or child may require a larger number of smaller breaths or puffs via the mouth and/or nose. The lower pressure and larger numbers of breaths administered to an infant or child may reduce the likelihood of injury to the respiratory system of the infant or child. Similarly, the force used in administering the chest compressions is reduced when administering CPR to an infant or child. Accordingly, a person who administers CPR must consider several variables and remember a variety of protocols.

CPR is more effective the sooner it is initiated and thus, the time between the onset of the medical emergency and the time of initiating CPR may be critical. Brain cells may begin to die in as little as 4-6 minutes without an adequate supply of oxygen. Unfortunately, medical emergencies can, and often do, happen at locations that are remote to medical facilities and where no trained medical professionals are readily available and, thus, a by-stander may be in the best position to perform CPR.

### BRIEF DESCRIPTION OF THE DRAWINGS

Advantages of the present invention will become apparent to those skilled in the art with the benefit of the following detailed description of embodiments and upon reference to the accompanying drawings in which:

FIG. 1 depicts a block diagram overview of an ARBU (automatic rescue breathing unit) system, according to some embodiments.

FIG. 2 depicts an isometric view representation of an ARBU device, according to some embodiments.

FIG. 3 depicts a top view representation of an ARBU device, according to some embodiments.

FIG. 4 depicts a front view representation of an ARBU device, according to some embodiments.

FIG. 5 depicts a back view representation of an ARBU device, according to some embodiments.

FIG. 6 depicts a perspective representation of an inlet adapter, according to some embodiments.

FIG. 7 depicts a perspective representation of a manifold, according to some embodiments.

FIG. 7A depicts a perspective representation of a pressure relief valve, according to some embodiments.

FIG. 8 depicts a perspective representation of an on/off valve, according to some embodiments.

FIG. 9 depicts a perspective representation of an on/off valve in a mounting assembly, according to some embodiments.

FIG. 10 depicts a perspective representation of a keyhole assembly, according to some embodiments.

FIG. 11 depicts a perspective representation of four keyhole assemblies with four keyholes, according to some embodiments.

FIG. 12 depicts a perspective representation of an adult key assembly, according to some embodiments.

FIG. 13 depicts a perspective representation of a child key assembly, according to some embodiments.

FIG. 14 depicts a perspective representation of an infant key assembly, according to some embodiments.

FIG. 15 depicts a perspective representation of a constant flow key assembly, according to some embodiments.

FIG. 16 depicts a side-view representation of an adult key on an adult key assembly inserted in a keyhole, according to some embodiments.

FIG. 16A depicts an enlarged view of a locking engagement engaged with a notch of a keyhole.

FIG. 17 depicts an example representation of a reset module, according to some embodiments.

FIG. 18 depicts an example representation of an on-delay timer, according to some embodiments.

FIG. 19 depicts a schematic representation of a pneumatic circuit to provide pulsed air flow with a reset module and on-delay timer, according to some embodiments.

FIG. 20 depicts an example representation of a pressure regulator, according to some embodiments.

FIG. 21 depicts an example representation of a needle valve, according to some embodiments.

FIG. 22 depicts an example representation of a check valve, according to some embodiments.

FIG. 23 depicts an example representation of an adapter, according to some embodiments.

FIG. 24 depicts an example representation of a system outlet, according to some embodiments.

FIG. 25 depicts a cross-sectional representation of a system outlet connected to an adapter, according to some embodiments.

FIG. 26 depicts an example representation of a keyhole with chamfered edges, according to some embodiments.

FIG. 27 depicts an example representation of implementation of an ARBU device in treatment of a patient, according to some embodiments.

FIG. 28 shows various embodiments for positioning a pressure relief valve in components associated with a key assembly.

FIG. 29 depicts an example representation of implementation of a compression pad with an airway device in treatment of a patient, according to some embodiments.

FIG. 29A depicts an example representation of implementation of a breath pause button with an airway device in treatment of a patient, according to some embodiments.

FIG. 30 depicts a block diagram overview of three separate ARBU devices intended for different types of patients, according to some embodiments.

While the invention may be susceptible to various modifications and alternative forms, specific embodiments thereof are shown by way of example in the drawings and will herein be described in detail. The drawings may not be to scale. It should be understood, however, that the drawings and detailed description thereto are not intended to limit the invention to the particular form disclosed, but to the contrary, the intention is to cover all modifications, equivalents, and alternatives falling within the spirit and scope of the present invention as defined by the appended claims.

### DETAILED DESCRIPTION OF EMBODIMENTS

When cardiac arrest occurs, the heart stops beating. The loss of heart function causes oxygen content, the amount of usable oxygen in the blood, to be depleted. Cardiopulmonary resuscitation (CPR) on the cardiac arrest victim is required immediately. CPR consists of delivering chest compressions and air/oxygen (rescue breaths) to the patient. The goal of CPR is for the return of spontaneous circulation of blood to the body. Developments have been made in automatic rescue breathing units (ARBUs) in the treatment of patients suffering cardiac arrest to increase the chances of survival. Even with the implementation of ARBUs, however, errors can be made, even by trained professionals, that may decrease the chances of survival. Errors may, for instance, be made in the rates and volumes of rescue breaths provided to the patient due to the complexities involved in remembering and delivering appropriate rates and volumes for different types (e.g., ages or sizes) of patients.

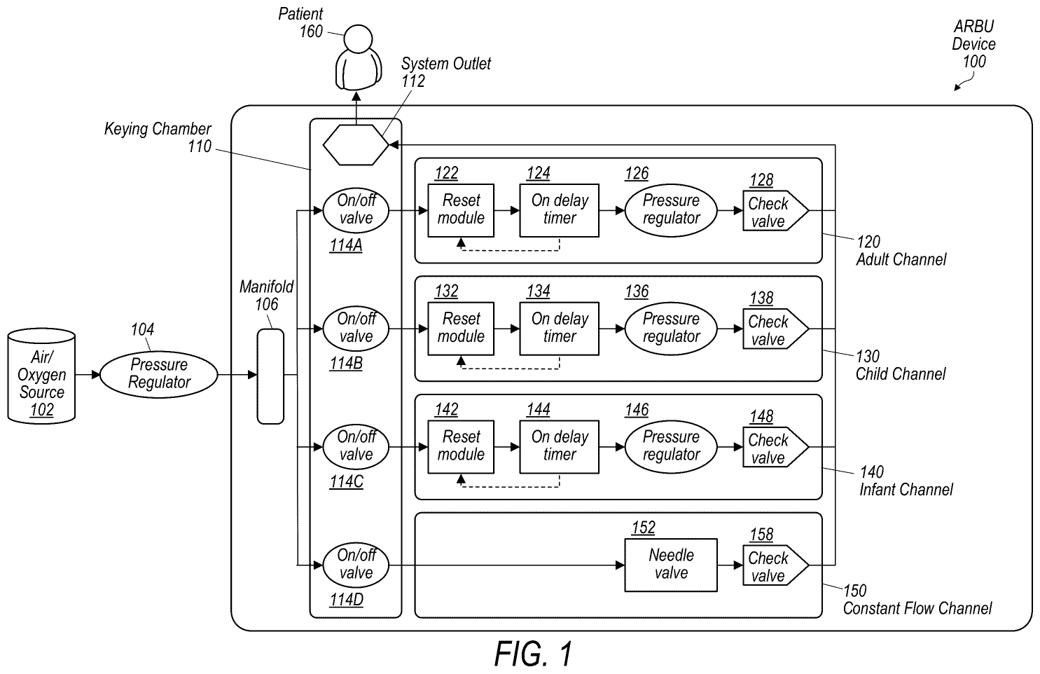

The present disclosure contemplates various embodiments of an automatic rescue breathing unit (ARBU) that includes two sub-systems—an airway device keying mechanism and a ventilation unit where the keying mechanism triggers and determines breath profile and parameters delivered by the ventilation unit. FIG. 1 depicts a block diagram overview of an ARBU system, according to some embodiments. In the illustrated embodiment, the ARBU system includes ARBU device 100 connected to air/oxygen source 102 via pressure regulator 104. ARBU device 100 includes manifold 106, keying chamber 110, adult channel 120, child channel 130, infant channel 140, and constant flow channel 150.

In various embodiments, device 100 is connected to patient 160 to provide oxygen/air flow to the patient. For instance, an airway device intended to be placed on the airway of patient 160 is coupled to system outlet 112 of keying chamber 110. As described herein, an “airway device” may include any of, but not be limited to, masks, endotracheal tubes, laryngeal mask airways, Igels, King airways, or any other airway device suitable for providing a flow or pulse of air to patient 160. In certain embodiments, system outlet 112 is the only air outlet on device 100. Having only a single outlet from device 100 may prevent undesired use of the device, as described herein. When device 100 is activated, compressed oxygen/air from air/oxygen source 102 (e.g., an oxygen tank) flows into manifold 106 through pressure regulator 104. Pressure regulator 104 controls a pressure of oxygen drawn into manifold 106. For example, pressure regulator 104 may limit the pressure to prevent overpressure in device 100. In one embodiment, pressure is regulated to a pressure of 50 psi through pressure regulator 104.

In certain embodiments, manifold 106 has five outlets (see, e.g., FIG. 7), four of which connect into keying chamber 110, with each outlet connected to one of the four channels (e.g., adult channel 120, child channel 130, infant channel 140, and constant flow channel 150) in device 100 by independently operated on/off valves 114A-D in keying chamber 110. The fifth outlet of manifold 106 may connect to a pressure relief valve (e.g., pressure relief valve 109, shown in FIG. 7A and described below). In various embodiments, on/off valve 114A corresponds to adult channel 120, on/off valve 114B corresponds to child channel 130, on/off valve 114C corresponds to infant channel 140, and on/off valve 114D corresponds to constant flow channel 150. Accordingly, activation of each channel is independently controlled by its corresponding on/off valve 114A-D in keying chamber 110. Depending on the size of the airway device coupled to system outlet 112, keying chamber 110 (e.g., the keying mechanism) may activate one of the four channels (e.g., one of adult channel 120, child channel 130, infant channel 140, or constant flow channel 150) to deliver the required breath profile to patient 160. For example, keying chamber 110 may open the on/off valve 114 for the channel to be activated and the remaining on/off valves will remain closed. Accordingly, the activated channel receives air flow from manifold 106 while air flow is inhibited to the other channels according to the airway device detected by the keying mechanism.

In various embodiments, device 100 has size dimensions that are at most 35 cm×25 cm×15 cm. Device 100 may also have a weight of at most 15 pounds (e.g., about 6.8 kg). Having smaller dimensions and/or weight may enable device 100 to be portable and more easily maneuverable by personnel. In certain embodiments, device 100 does not include any electric components. Not having electric components in device 100 may eliminate electrical shock hazard when using the device. Further, device 100 may not have any knobs or other adjustment controls on the device that allow user adjustment of the device. Removing user adjustments may, in such embodiments, inhibit undesirable changes to the operating parameters of device 100.

As described above, the flow/respiratory parameters for device 100 may be determined based on the airway device size coupled to the device. The flow/respiratory parameters may be determined by rescue breath parameters defined by the American Heart Association. Table I provides example flow/respiratory parameters to be provided by device 100 according to airway device size.

TABLE I

| | | Tidal | Peak | |
|---|---|---|---|---|
| Airway Device | Flow Rate | Volume | Pressure | Respiration |
| Size | (L/min)* | (cc) | (cm of H₂O) | Rate (bpm)** |
| Adult | 30 ± 10 | 600 ± 10% | 40 ± 10% | 10 |
| Child | 15 ± 5 | 300 ± 10% | 20 ± 10% | 20 |
| Infant | 5 ± 2 | 100 ± 10% | 20 ± 10% | 30 |
| Constant Flow | 15 | — | — | — |

*Flow rate is a function of tidal volume and rate
**There is intended to be no fluctuations in respiration rate

In TABLE I, the peak pressure refers to the maximum air pressure in the chest cavity to which the patient will be exposed. Tidal volume refers to the volume of air/oxygen that will be provided to the patient during each inhalation and the respiratory rate refers to the number of breaths that will be provided to the patient by device 100 over a period of time (e.g., over a minute).

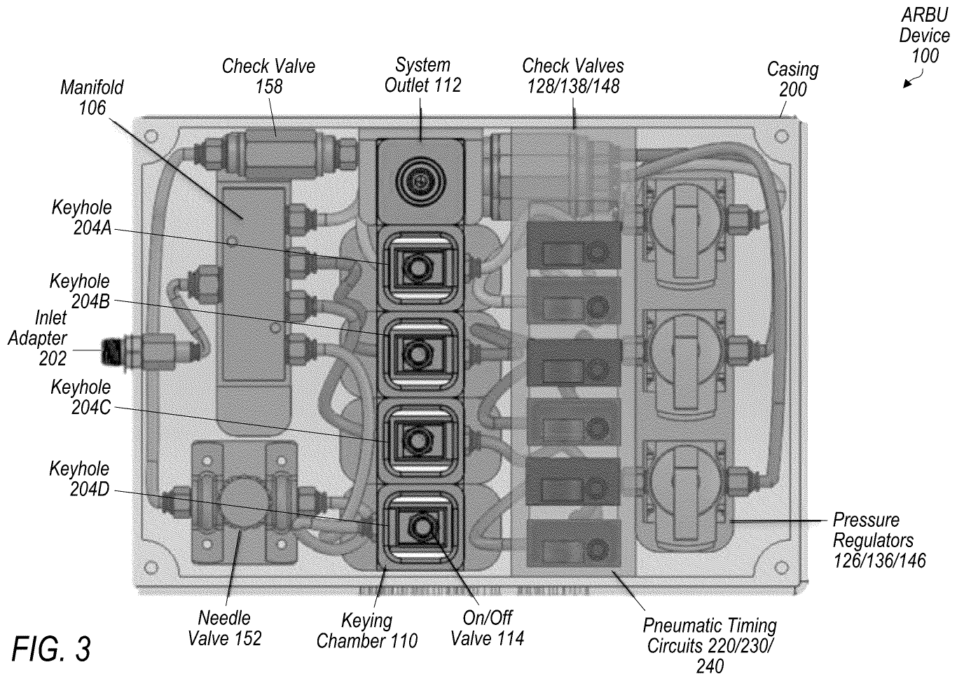

For the illustrated embodiment of FIG. 1, adult channel 120 is configured to provide flow/respiratory parameters corresponding to the adult airway device size in TABLE I, child channel 130 is configured to provide flow/respiratory parameters corresponding to the child airway device size in TABLE I, infant channel 140 is configured to provide flow/respiratory parameters corresponding to the infant airway device size in TABLE I, and constant flow channel 150 is configured to provide flow/respiratory parameters corresponding to the constant flow airway device size in TABLE I. As shown in FIG. 1, each of adult channel 120, child channel 130, and infant channel 140 includes a pneumatic timing circuit with a reset module (122, 132, and 142 in the channels, respectively) and an on-delay pneumatic timer (124, 134, 144 in the channels, respectively). In the channels, pulsed oxygen outputs from the pneumatic timing circuit (220, 230, and 240 in the channels, respectively, shown in FIG. 3) are passed into a pressure regulator (126, 136, and 146 in the channels, respectively). The pressure regulators control the input pressure to regulate the input pressure and convert it to the flow rate needed for the specific channel. For instance, an input flow rate pressure of 50 psi may be reduced to a flow rate of 6 L/min in adult channel 120 by pressure regulator 126. After the downregulation, the air/oxygen is passed through a check valve (128, 138, and 148 in the channels, respectively) and then to system outlet 112.

In certain embodiments, constant flow channel 150 includes needle valve 152 and check valve 158. Needle valve 152 is used to control the flow rate through constant flow channel 150 without any pressure regulation. In various embodiments, components in the channels are coupled using pneumatic tubing (e.g., 6 mm pneumatic tubing) with NPT push-to-connect adapters for fittings to the components. Teflon tape or another sealing material may also be used on the adapters to inhibit air leakage in device 100.

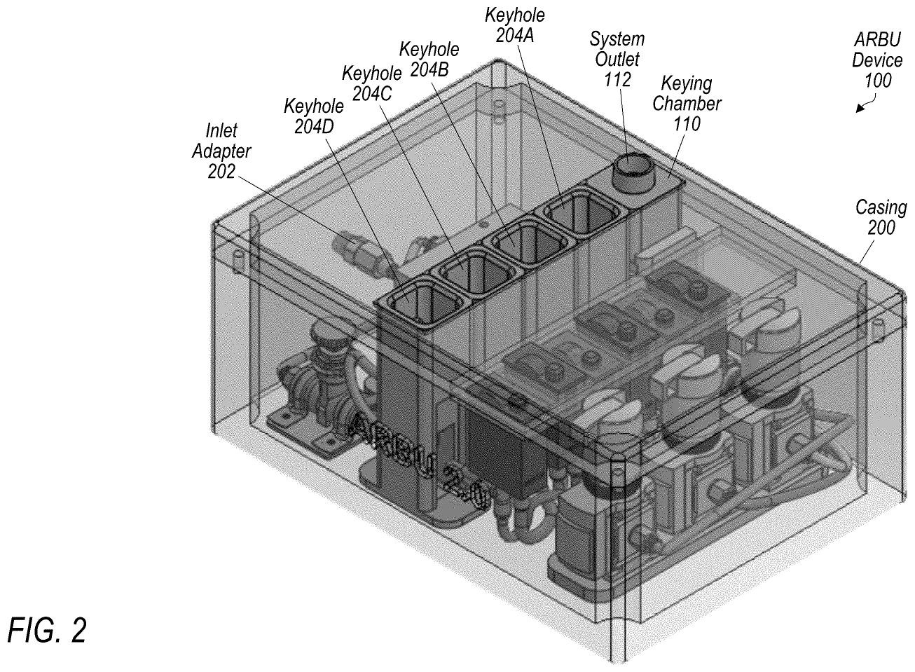

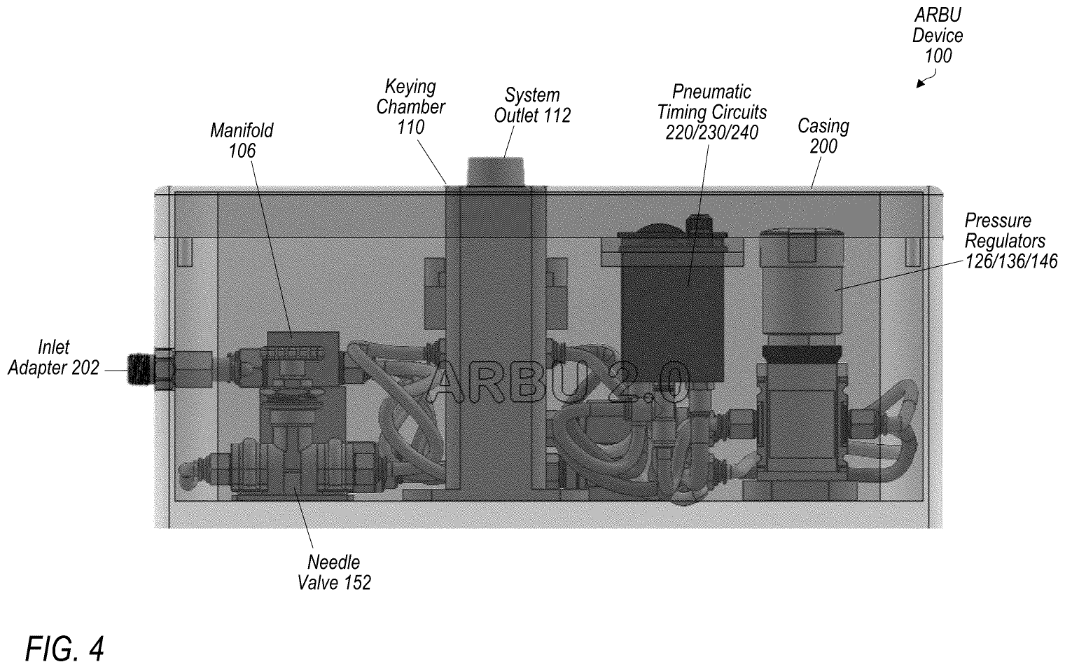

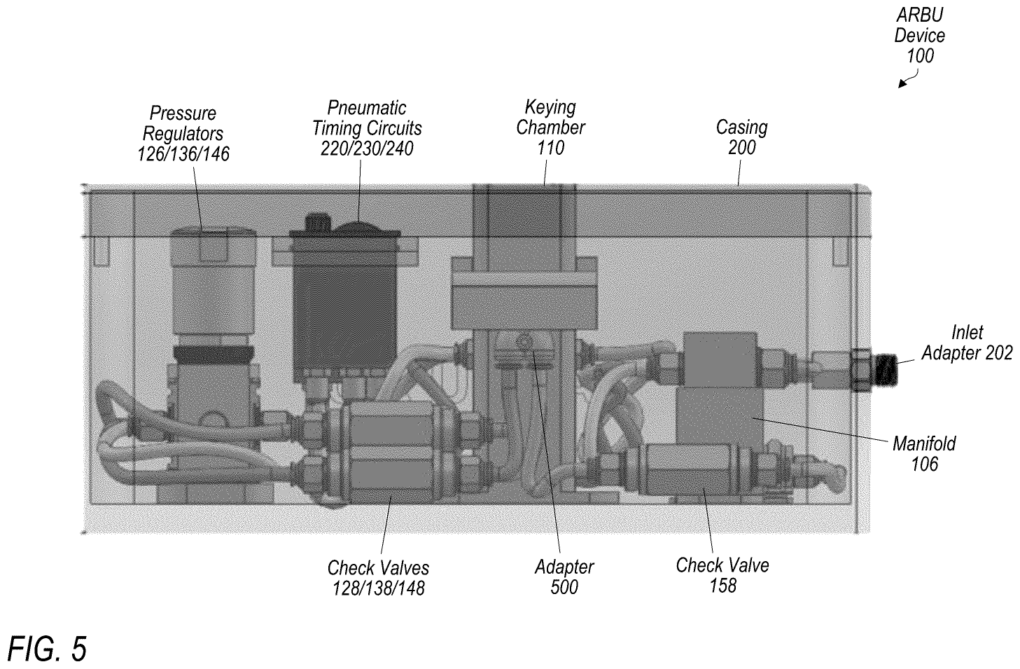

FIG. 2 depicts an isometric view representation of device 100, according to some embodiments. FIG. 3 depicts a top view representation of device 100, according to some embodiments. FIG. 4 depicts a front view representation of device 100, according to some embodiments. FIG. 5 depicts a back view representation of device 100, according to some embodiments. In the illustrated embodiments, the components of device 100 are located inside casing 200 (which is transparent in the drawings). Inlet adapter 202 is connected to manifold 106 to provide an air/oxygen inlet to device 100. For example, inlet adapter 202 may connect manifold 106 to pressure regulator 104, shown in FIG. 1.

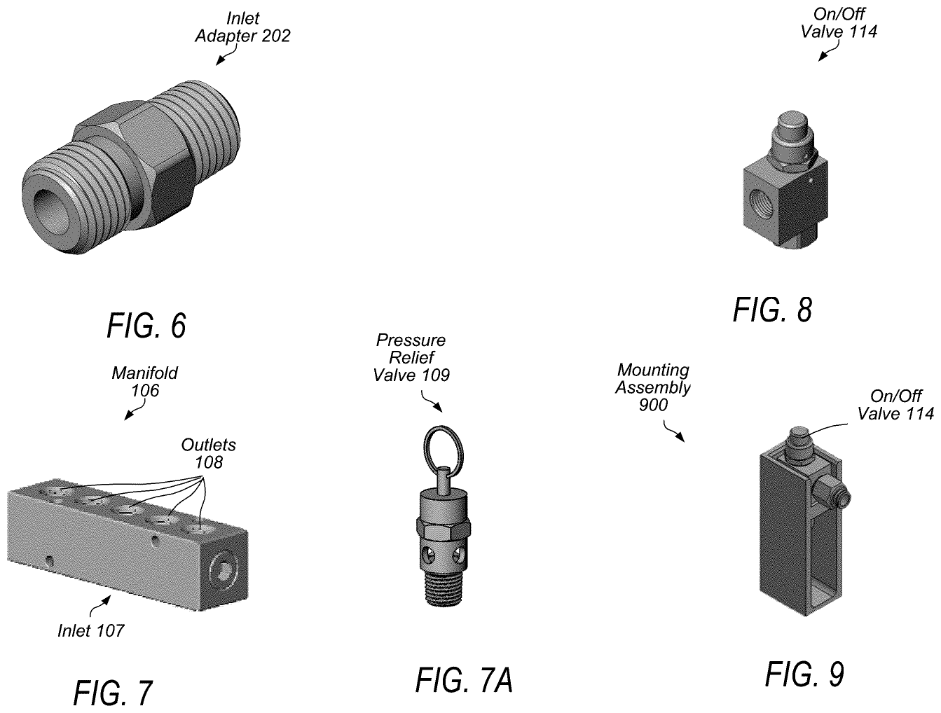

FIG. 6 depicts a perspective representation of inlet adapter 202, according to some embodiments. In various embodiments, inlet adapter 202 is designed to protrude out of casing 200 of device 100, as shown in FIG. 3. In certain embodiments, inlet adapter 202 is a hose fitting designed for compressed gas. For example, in one embodiment, inlet adapter 202 is a 6 mm outside diameter×¼ NPT push-to-connect adapter.

FIG. 7 depicts a perspective representation of manifold 106, according to some embodiments. Air/oxygen may enter manifold 106 through inlet adapter 202 and pressurize the manifold. In certain embodiments, manifold 106 includes one inlet (e.g., inlet 107 connected to inlet adapter 202) and five outlets 108. In one embodiment, four outlets 108 are connected to on/off valves 114A-D and one outlet 108 is connected to pressure relief valve 109. FIG. 7A depicts a perspective representation of pressure relief valve 109, according to some embodiments, that may be connected to one outlet 108 of manifold 106. Pressure relief valve 109 may be coupled to manifold 106 to inhibit excessive pressure in device 100. In some embodiments, the inlet of manifold 106 is a female port with ⅜ NPT threading and the outlets are female ports with ¼ NPT threading. The inlet/outlets of manifold 106 may be connected to push-to-connect fittings. In certain embodiments, the inlet is on one side of manifold 106 and the outlets are on the opposite side of the manifold. Having the inlet/outlets on opposite sides allows air/oxygen to flow in a straight path and improve air flow through device 100. In one embodiment, manifold 106 is aluminum (e.g., anodized aluminum).

In various embodiments, the outlets of manifold 106 are connected to on/off valves 114A-D in keying chamber 110, in addition to pressure relief valve 109. FIG. 8 depicts a perspective representation of on/off valve 114, according to some embodiments. In certain embodiments, on/off valve 114 is a push button valve where the valve is normally off (e.g., off when the button is not pushed). Thus, when an airway device key (described below) pushes on the button, on/off valve 114 is opened to allow air to flow through the valve. For instance, on/off valve 114 may include a spring that normally closes off the valve until the button is pushed down (e.g., depressed or activated). Accordingly, when the button is pushed down (e.g., activated) to overcome the force of the spring, air flows through the valve. When the button is released and moves upwards, the spring closes the valve and closes off air flow through the valve. On/off valve 114 may include an exhaust port through which air is released when closed to inhibit over pressurization of the valve.

In various embodiments, on/off valve 114 is placed in a mounting assembly for placement in keying chamber 110. FIG. 9 depicts a perspective representation of on/off valve 114 in mounting assembly 900, according to some embodiments. Mounting assembly 900 may be, for example, a 3D printed mounting assembly. In some embodiments, mounting assembly 900 is configured to be mounted to a base of casing 200 or another rigid structure. Mounting of mounting assembly 900 may ensure stability of on/off valve 114 during operation of the valve (e.g., during pushing of the valve to open the valve).

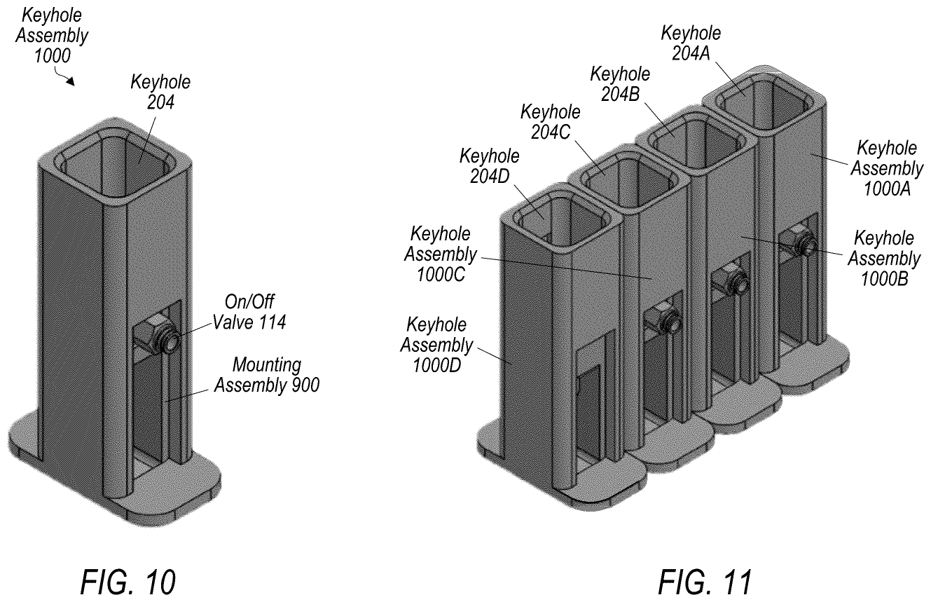

In certain embodiments, mounting assembly 900, with on/off valve 114, is placed inside a keyhole assembly for positioning in keying chamber 110. FIG. 10 depicts a perspective representation of keyhole assembly 1000, according to some embodiments. In the illustrated embodiment, mounting assembly 900 is positioned (with on/off valve 114) in a lower portion of keyhole assembly 1000. Keyhole assembly 1000 may be, for example, a 3D printed assembly. Keyhole assembly 1000 includes keyhole 204 that allows access to the push button of on/off valve 114. Keyhole 204 may, for instance, allow a key (described below) to be inserted and activate (e.g., depress) the push button of on/off valve 114.

FIG. 11 depicts a perspective representation of four keyhole assemblies 1000A-D with four keyholes 204A-D, according to some embodiments. The four keyholes 204A-D, shown in FIG. 11, correspond to the four keyholes 204A-D, shown in FIGS. 2 and 3. Each of the keyholes 204A-D provides access to an on/off valve 114A-D that corresponds to one of the channels (e.g., one of adult channel 120, child channel 130, infant channel 140, or constant flow channel 150). For instance, keyhole 204A and on/off valve 114A may correspond to adult channel 120, keyhole 204B and on/off valve 114B may correspond to child channel 130, keyhole 204C and on/off valve 114C may correspond to infant channel 140, and keyhole 204D and on/off valve 114D may correspond to constant flow channel 150.

With keyholes 204A-D arranged as shown in FIGS. 2 and 3, various embodiments may be contemplated where key assemblies (which may be 3D printed) are designed to connect to system outlet 112 and activate an on/off valve 114 within keyhole 204 that corresponds to the key assembly 1000. Thus, a specific key assembly 1000 may be designed to be attached to an airway device with a specific intended use (e.g., use for an adult, a child, an infant, or constant flow) and the specific key assembly may engage only with the keyhole 204 and on/off valve 114 associated with the airway device's intended use. For example, an adult key assembly attached to an adult airway device may only engage keyhole 204A, which is associated with adult channel 120, such that only the on/off valve 114A for the adult channel is activated when using the adult key assembly.

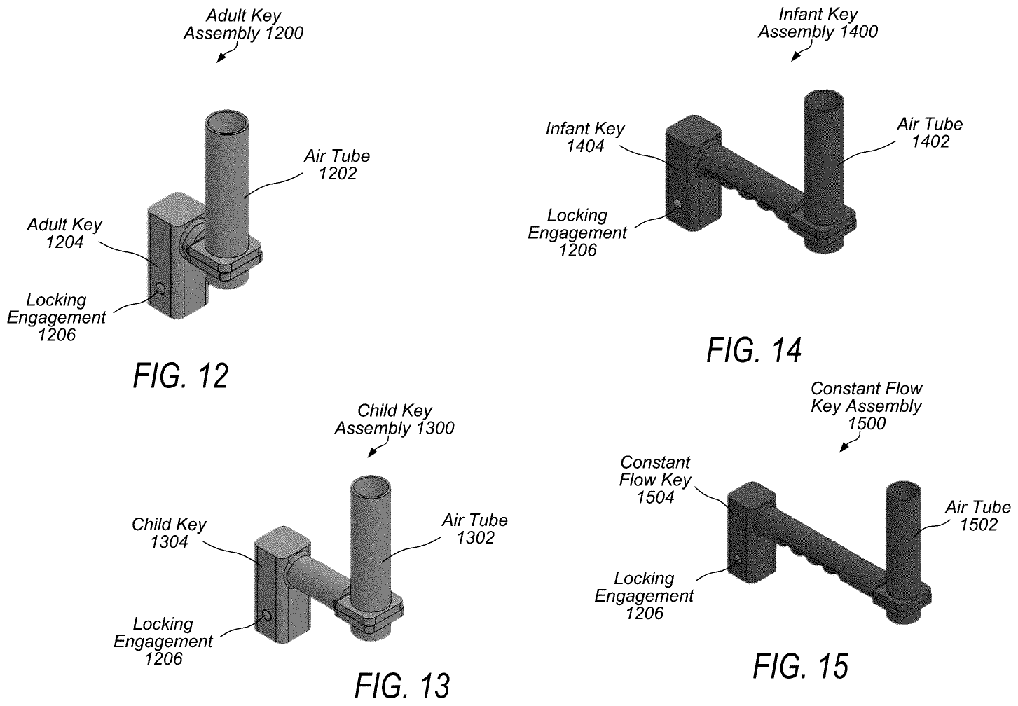

FIG. 12 depicts a perspective representation of adult key assembly 1200, according to some embodiments. In the illustrated embodiment, adult key assembly 1200 includes air tube 1202 and adult key 1204. Air tube 1202 is configured to be attached to system outlet 112 in keying chamber 110. When air tube 1202 is attached to system outlet 112, key 1204 is configured to engage the on/off valve 114A through keyhole 204A. For instance, the distance between air tube 1202 and key 1204 is defined such that when air tube 1202 is connected to system outlet 112, the key fits into keyhole 204A and engages on/off valve 114A. In certain embodiments, when air tube 1202 is fully connected to system outlet 112, key 1204 depresses the push button of on/off valve 114A and opens the valve to allow air flow through adult channel 120. When adult key assembly 1200 is removed, on/off valve 114A closes again and cuts off air flow through adult channel 120. In some embodiments, adult key assembly 1200 includes locking engagement 1206, which is discussed in more detail below.

FIG. 13 depicts a perspective representation of child key assembly 1300, according to some embodiments. In the illustrated embodiment, child key assembly 1300 includes air tube 1302 and child key 1304. The distance between air tube 1302 and child key 1304 is defined such that when air tube 1302 is connected to system outlet 112, the child key fits into keyhole 204B and engages on/off valve 114B. Thus, when air tube 1302 is fully connected to system outlet 112, child key 1304 depresses the push button of on/off valve 114B and opens the valve to allow air flow through child channel 130. When child key assembly 1300 is removed, on/off valve 114B closes again and cuts off air flow through child channel 130. In some embodiments, child key assembly 1300 includes locking engagement 1206, which is discussed in more detail below.

FIG. 14 depicts a perspective representation of infant key assembly 1400, according to some embodiments. In the illustrated embodiment, infant key assembly 1400 includes air tube 1402 and infant key 1404. The distance between air tube 1402 and infant key 1404 is defined such that when air tube 1402 is connected to system outlet 112, the infant key fits into keyhole 204C and engages on/off valve 114C. Thus, when air tube 1402 is fully connected to system outlet 112, infant key 1404 depresses the push button of on/off valve 114C and opens the valve to allow air flow through infant channel 140. When infant key assembly 1400 is removed, on/off valve 114C closes again and cuts off air flow through infant channel 140. In some embodiments, infant key assembly 1400 includes locking engagement 1206, which is discussed in more detail below.

FIG. 15 depicts a perspective representation of constant flow key assembly 1500, according to some embodiments. In the illustrated embodiment, constant flow key assembly 1500 includes air tube 1502 and constant flow key 1504. The distance between air tube 1502 and constant flow key 1504 is defined such that when air tube 1502 is connected to system outlet 112, the constant flow key fits into keyhole 204D and engages on/off valve 114D. Thus, when air tube 1502 is fully connected to system outlet 112, constant flow key 1504 depresses the push button of on/off valve 114D and opens the valve to allow air flow through constant flow channel 150. When constant flow key assembly 1500 is removed, on/off valve 114D closes again and cuts off air flow through constant flow channel 150. In some embodiments, constant flow key assembly 1500 includes locking engagement 1206, which is discussed in more detail below.

As shown by the various embodiments of key assemblies depicted in FIGS. 12-15, the keys can have defined distances from the air tube that connects to system outlet 112 such that, when a particular key assembly is utilized, only a specific channel associated with the key assembly is opened to air flow. Thus, with the specific key assemblies being designated for specific types of airway devices (e.g., adult key assembly 1200 for an adult airway device, child key assembly 1300 for a child airway device, infant key assembly 1400 for an infant airway device, and constant flow key assembly 1500 for a constant flow airway device), selection of the proper channel flow for air through device 100 automatically occurs. In other words, when a user selects an airway device (e.g., an adult airway device) with the appropriate key assembly already attached to the airway device (e.g., adult key assembly 1200), the proper air channel (e.g., adult channel 120) is automatically selected when the user attaches the key assembly to system outlet 112 on device 100. In various embodiments, key assemblies 1200, 1300, 1400, 1500 include ergonomic features or textured features to case handling of the key assemblies. These features may also provide indication to personnel of the differences between the key assemblies.

Automatic selection of the proper air channel as described herein provides a safe and secure method for selection of the proper air channel and its associated flow/respiratory parameters (as shown in TABLE I) based on selection of an airway device. Giving CPR and providing assisted air is often done in a high stress situation where humans can easily make mistakes. Device 100, with its automatic selection of air channel and flow/respiratory parameters, mitigates many risks associated with human decision-making and makes for a safer and more reliable administration of life-saving techniques.

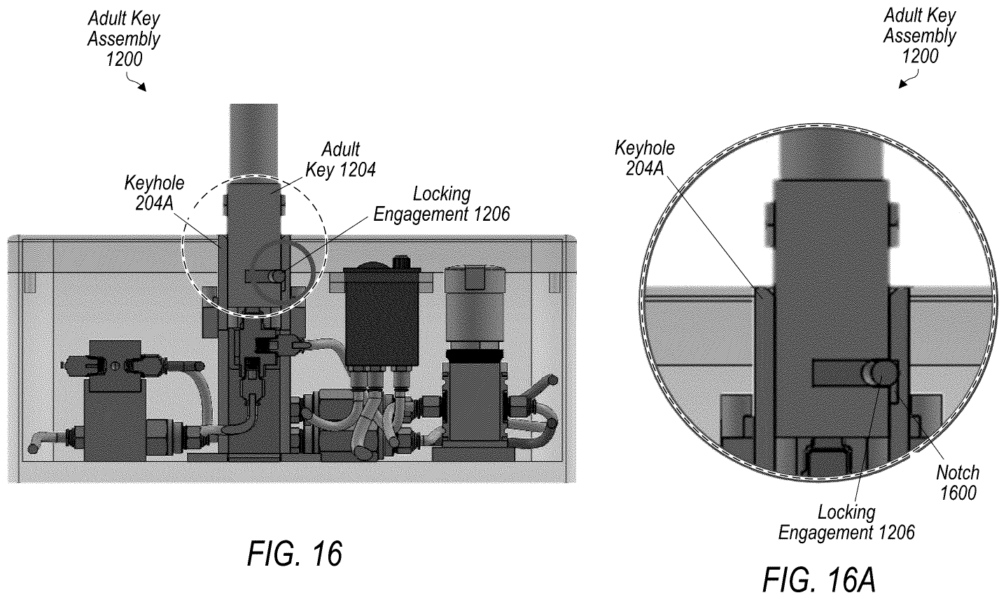

In some embodiments, as shown in FIGS. 12-15, a key assembly includes locking engagement 1206. Locking engagement 1206 may be, for example, a spring-ball detent press-fit into the key or another engagement component that is part of a locking mechanism to secure the key assembly in a keyhole. FIG. 16 depicts a side-view representation of adult key 1204 on adult key assembly 1200 inserted in keyhole 204A, according to some embodiments. FIG. 16A depicts an enlarged view of locking engagement 1206 engaged with notch 1600 of keyhole 204A. Locking engagement 1206 and notch 1600 may together form the locking mechanism for the key assembly.

In some embodiments, as adult key 1204 is inserted into keyhole 204A, locking engagement 1206 (e.g., the ball) is pushed inwards on the key and then pops back out when the locking engagement 1206 engages notch 1600. As shown in FIGS. 16 and 16A, when key assembly 1200 and key 1204 is fully inserted in keyhole 204A, locking engagement 1206 engages notch 1600 and the key is secured in the keyhole. The engagement between locking engagement 1206 and notch 1600 may be overcome by a small force imparted by the user when the user wants to disengage key assembly 1200 from device 100. The locking mechanism provided by locking engagement 1206 and notch 1600 may inhibit accidental displacement of the key assembly and its air tube during use of device 100. In various embodiments, key assemblies are made of materials suitable for repeated insertion and removal of the key assemblies. For instance, the key assemblies may be made of carbon fiber infused nylon or other high strength, low weight materials.

As described above, adult channel 120, child channel 130, and infant channel 140 have pneumatic timing circuits (shown in FIG. 3) associated with the channels. For instance, adult channel 120 is associated with pneumatic timing circuit 220, child channel 130 is associated with pneumatic timing circuit 230, and infant channel 140 is associated with pneumatic timing circuit 240. Pneumatic timing circuits 220, 230, 240 are implemented to control the breathing parameters (e.g., breaths per minute and/or breath volume) provided by each of the channels 120, 130, 140, respectively. A pneumatic timing circuit may include a reset module and an on-delay timer.

Accordingly, in certain embodiments, pneumatic timing circuit 220 includes reset module 122 and on-delay timer 124 in adult channel 120, pneumatic timing circuit 230 includes reset module 132 and on-delay timer 134 in child channel 130, and pneumatic timing circuit 240 includes reset module 142 and on-delay timer 144 in infant channel 140. Pneumatic timing circuits 220, 230, 240 may operate to provide the flow/respiratory parameters listed in TABLE I for each of channels 120, 130, 140, respectively. The reset modules and on-delay timers work in combination to provide pulsed air flow through the channels with the proper parameters.

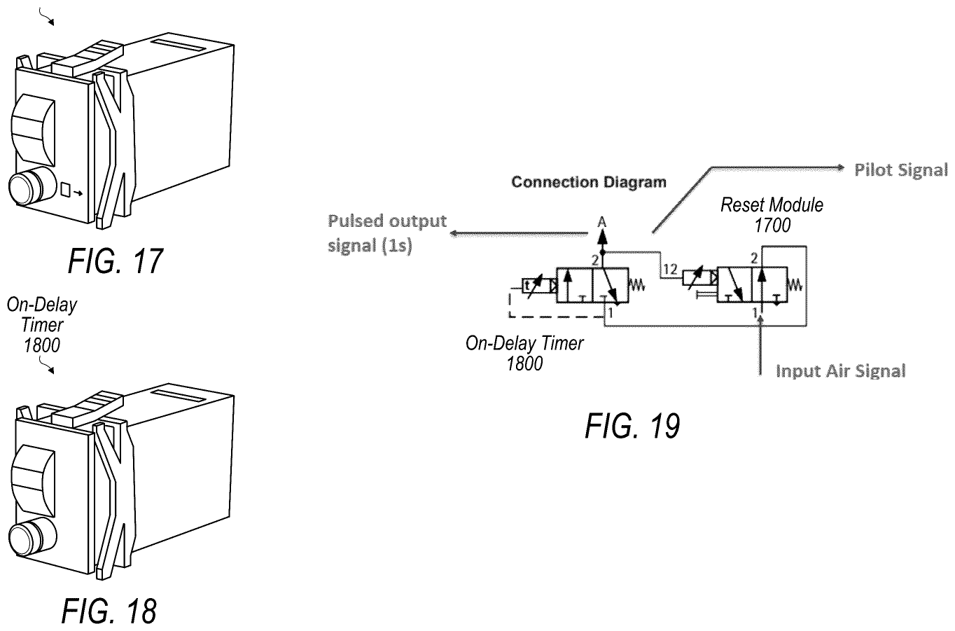

FIG. 17 depicts an example representation of a reset module 1700, according to some embodiments. FIG. 18 depicts an example representation of an on-delay timer 1800, according to some embodiments. Both reset module 1700 and on-delay timer 1800 may be components made by Impulse Automation Ltd (Andover, United Kingdom). FIG. 19 depicts a schematic representation of a pneumatic circuit to provide pulsed air flow with a reset module and on-delay timer, according to some embodiments. In various embodiments, reset module 1700 is used to reset on-delay timer 1800 to create a continuous timing circuit.

In the illustrated embodiment of the pneumatic circuit, input air is connected to port 1 of reset module 1700 and, in response, on-delay timer 1800 begins to count the time (on-delay timer 1800 receiving air from port 2 of reset module 1700 through port 1 on the on-delay timer). Air is then passed from port 1 to port 2 of on-delay timer 1800, which is connected to port 12 of reset module 1700 as a pilot signal through a tube splitter on timeout. As one example, for a pulsed output of 1 second, after an output of 1 second (A), the air supply from port 2 of reset module 1700 to port 1 of on-delay timer 1800 is interrupted for 300 milliseconds, which resets on-delay timer 1800 and reset module 1700. This cycle will run continuously for as long as air is connected (e.g., supplied) to port 1 of reset module 1700. It should be noted that the timing may vary for different breath profiles (e.g., different timing may be implemented for an adult breath profile and a child breath profile).

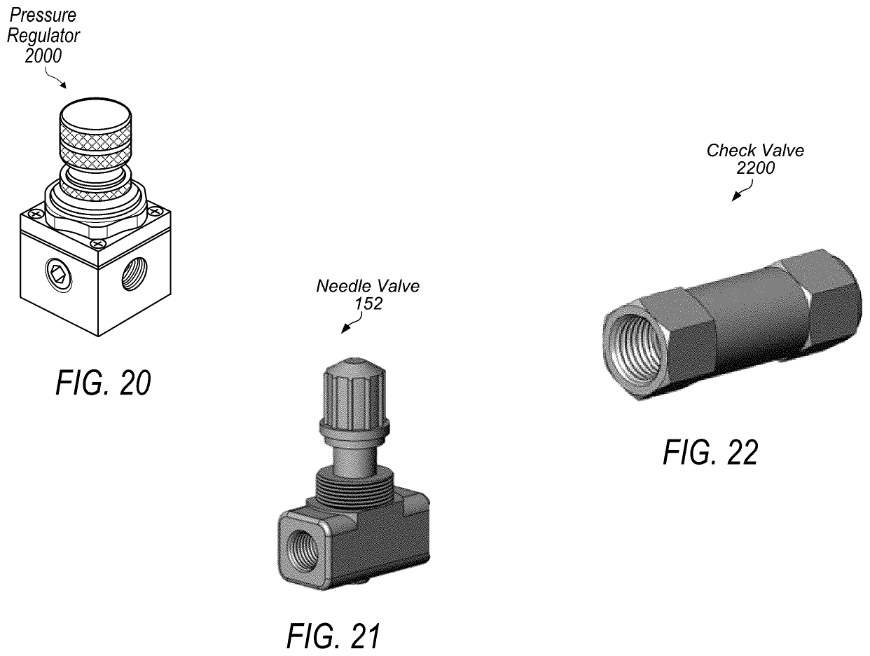

After the pneumatic timing circuits, air in channels 120, 130, 140 flows through a pressure regulator (e.g., pressure regulator 126, 136, 146, respectively, in the channels). The pressure regulator brings down the pressure to the desired value for its associated channel. FIG. 20 depicts an example representation of a pressure regulator 2000, according to some embodiments. Pressure regulator 2000 may be, for example, a pressure regulator manufactured by Ellis/Kuhnke Controls (Eatontown, New Jersey).

In various embodiments, constant flow channel 150 includes needle valve 152, as shown in FIG. 1. FIG. 21 depicts an example representation of a needle valve 152, according to some embodiments. Needle valve 152 may be, for example, a needle valve obtained from McMaster-Carr Supply Company (Elmhurst, Illinois). Needle valve 152 may be chosen for providing the constant flow of air as a needle valve is a pneumatic device that can provide a constant flow rate. For instance, needle valve 152 may include a tapered pin that gradually opens a chamber to provide a fine flow of air. In certain embodiments, needle valve 152 includes a knob that is set to tune the flow rate of air passing through the valve. Accordingly, needle valve 152 may be set to provide a constant flow rate of oxygen at the input pressure. In certain embodiments, needle valve 152 provides a constant flow rate of 15 L/min at an input pressure of 50 psi. In various embodiments, the knob on needle valve 152 may be locked in position after assembly of device 100 to inhibit changing of the flow rate of air passing through the valve from a determined setting.

In certain embodiments, all the channels include check valves though embodiments may be contemplated with some channels not having check valves. For instance, adult channel 120 includes check valve 128, child channel 130 includes check valve 138, infant channel 140 includes check valve 148, and constant flow channel 150 includes check valve 158, as shown in FIG. 1. FIG. 22 depicts an example representation of a check valve 2200, according to some embodiments. Check valve 2200 may be, for example, a check valve obtained from McMaster-Carr Supply Company (Elmhurst, Illinois). Check valves are unidirectional valves that may be placed at the ends of channels to ensure air/oxygen only flows in the intended direction. Accordingly, check valves may be utilized to ensure that no backflow of oxygen into device 100 occurs. In one embodiment, the check valves are brass and have a minimum opening pressure of 0.3 psi.

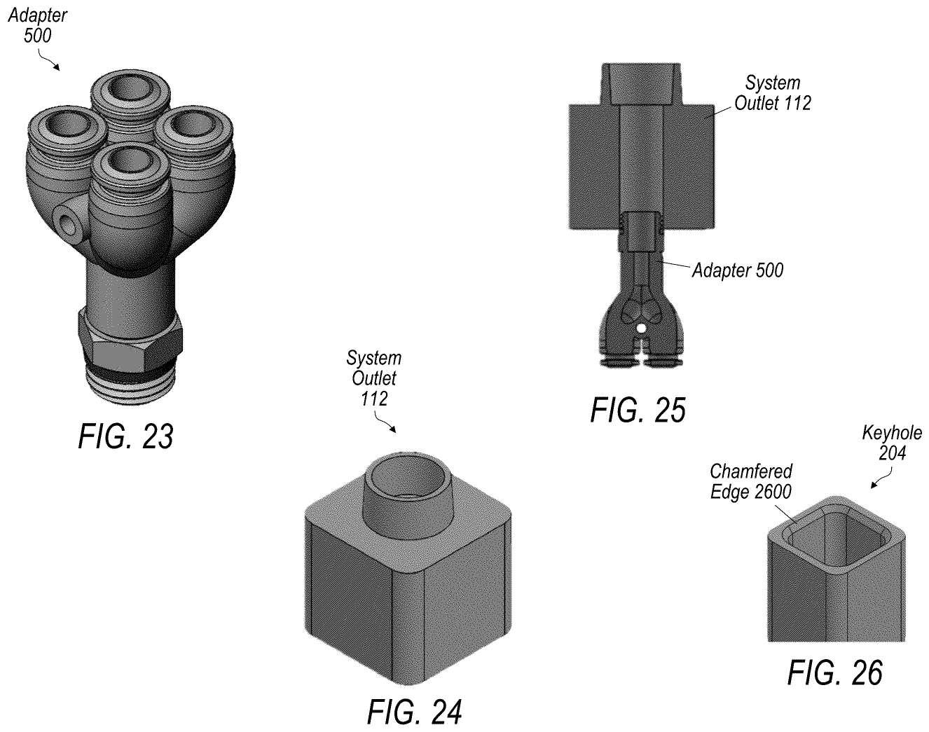

As shown in FIG. 5, after check valves 128, 138, 148, and 158, multiple air lines enter keying chamber 110. These multiple air lines are connected to system outlet 112, which is a single outlet. In certain embodiments, adapter 500 is used to combine the multiple air lines into a single air line connected to system outlet 112. FIG. 23 depicts an example representation of adapter 500, according to some embodiments. Adapter 500 may be made of nylon or another air impermeable material. In certain embodiments, adapter 500 is a double-wye adapter with 4 inlets connected to 1 outlet to combine the flow from the 4 inlets into a single outlet flow. The inlets may be connectable to pneumatic tubing coming from check valves while the outlet may include universal threading for connecting to NPT and NPTF threads. The use of the double-wye configuration for adapter 500 allows straight line flow of pulsed oxygen signals, which requires no pressurization as would be the case for the utilization of a manifold. Adapter 500 may also utilize various gaskets and/or O-rings to inhibit air leakage from the connections to the adapter.

FIG. 24 depicts an example representation of system outlet 112, according to some embodiments. System outlet 112 may be, for example, a 3D printed nylon outlet. In some embodiments, system outlet is made of solid filled carbon infused nylon. System outlet 112 has a female connector on its lower end configured to connect to the male outlet of adapter 500. FIG. 25 depicts a cross-sectional representation of system outlet 112 connected to adapter 500, according to some embodiments. The various tubes and fittings utilized in device 100 include tubes and fittings suitable for pneumatic operation and/or a flow of air/oxygen. For instance, many tubes and fittings may be made of polyurethane rubber or other impermeable materials to inhibit leakage of oxygen from device 100.

In some contemplated embodiments, the edges of a keyhole (e.g., keyhole 204) are chamfered. FIG. 26 depicts an example representation of keyhole 204 with chamfered edges 2600, according to some embodiments. Chamfered edges 2600 may inhibit damage (e.g., chipping) to the edges of keyhole 204 during repeated use of device 100 (e.g., during repeated insertion and removal of a key).

As described herein, device 100 is a completely pneumatic device without any electrical components. Accordingly, device 100 may not be susceptible to water damage or damage from other harsh environments that can damage electrical equipment. Additionally, casing 200 may be chemically sealed to inhibit contamination of the interior of device 100 and its components.

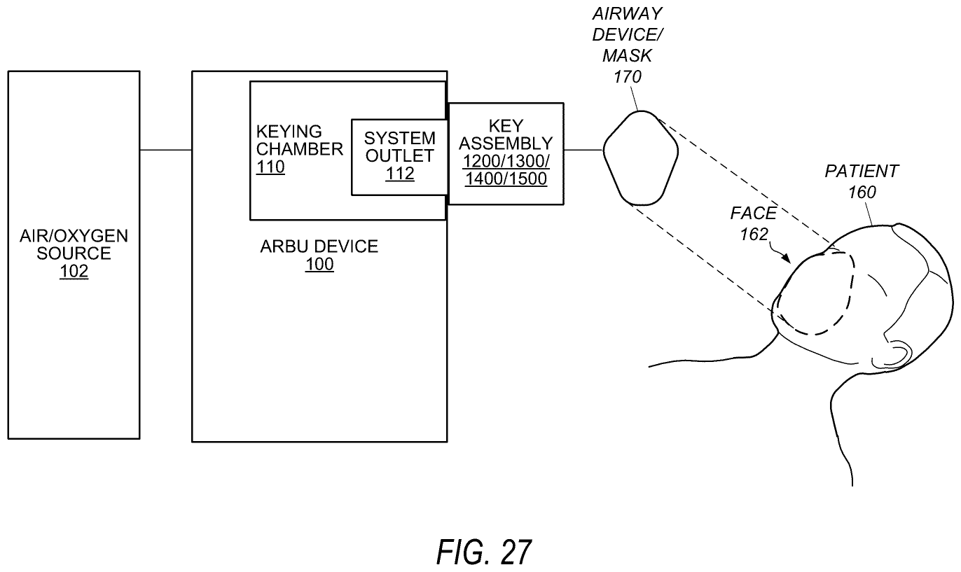

FIG. 27 depicts an example representation of implementation of ARBU device in treatment of patient 160, according to some embodiments. In the illustrated embodiment, device 100 is coupled to air/oxygen source 102. Device 100 is being implemented in the treatment of patient 160, shown with face 162. In various embodiments, device 100 is a portable system that is transported to a location of patient 160.

In various embodiments, source 102 is a source capable of providing pressurized air/oxygen for use by device 100. As used herein “pressurized air/oxygen” refers to air/oxygen having a pressure that promotes the flow of the air/oxygen from device 100 into the lungs of patient 160. In some embodiments, pressurized air/oxygen may have a pressure that is above a minimum-pressure threshold, such as 20 centimeters of water (cmH₂O) above ambient air pressure.

In certain embodiments, source 102 is a cylinder containing pressurized air/oxygen. The pressure of the air/oxygen may be set significantly above the minimum-pressure threshold such that the air/oxygen in the cylinder is maintained above the minimum-pressure threshold as the air/oxygen is expelled from the cylinder and the pressure of the air/oxygen in the source 102 drops as a function of the air/oxygen expelled from the cylinder. Source 102 may include a mechanical device, such as a compressor, configured to move and/or pressurize the air/oxygen. Such a mechanical device may be used to pressurize and/or fill a cylinder of the source 102. In one embodiment, source 102 may include the mechanical device to move the air from the cylinder to the subject.

During treatment (e.g., resuscitation) of patient 160, airway device 170 (e.g., a mask) is coupled to face 162, or applied to the airway, of the patient. As shown in FIG. 27, key assembly (e.g., one of key assemblies 1200, 1300, 1400, or 1500) is attached to airway device 170. For instance, as described herein, a specific key assembly is attached to a specific airway device/mask based on the type of airway device. The key assembly is then attached to system outlet 112 of keying chamber 110. Additionally, as described herein, key assembly further engages its corresponding keyhole and corresponding on/off valve to provide air flow through the correct channel in device 100.

### ADDITIONAL COMPONENT EMBODIMENTS

### Pressure Relief Valve

As described herein, ARBU device 100 is capable of providing multiple ventilation parameters (e.g., flow/respiratory parameters) that are predetermined and set for the device. Selection of an airway device and attached key assembly determines the air channel and its associated ventilation parameters are provided to the airway device. While the use of key assemblies provides safety for operation of device 100 by inhibiting the wrong ventilation parameters being provided through the wrong airway device, various additional embodiments may be contemplated to increase safety in operation of device 100 or a similar ARBU device.

In various embodiments, a pressure relief valve may be implemented to inhibit excessive pressure from being delivered to the lungs or other anatomical structures of the patient. In many instances, the pressure relief valve has been implemented in the ventilation unit itself when the ventilation unit has a single ventilation path through the unit. Device 100, however, has multiple air channels and thus may need multiple pressure relief valves, one for each air channel. Separate pressure relief valves may be needed for each channel due to the differences in air flow through the different air channels. Having multiple pressure relief valves may adversely increase the size and complexity of device 100.

To reduce or eliminate the need for pressure relief valves within device 100 itself, the present disclosure contemplates embodiments where the pressure relief valves are moved outside of the device. In certain embodiments, separate pressure relief valves are associated with, in some manner, independent key assemblies and airway devices utilized with device 100. Accordingly, much like a specific key assembly selects a specific air channel based on the airway device associated with the key assembly, a specific pressure relief valve may be provided for the specific air channel based on the pressure relief valve being attached to the key assembly.

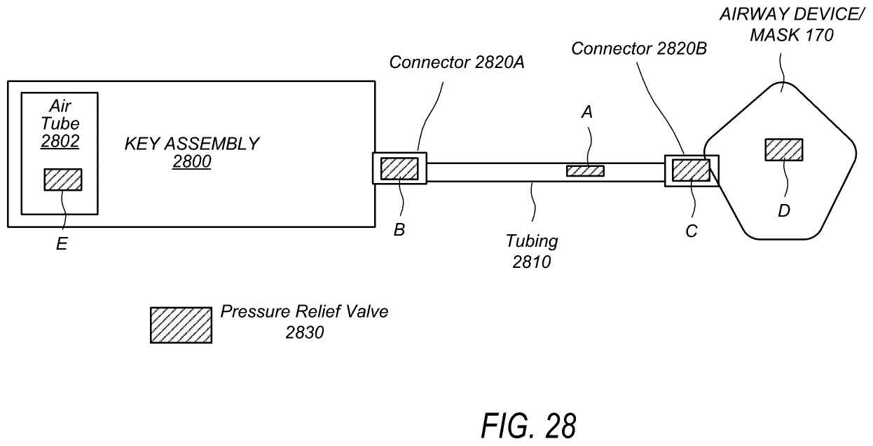

In various embodiments, a pressure relief valve may be associated with a key assembly by placing the pressure relief valve in components associated with the key assembly. For example, the pressure relief valve may be placed in components such as, but not limited to, tubing, a connector, or a mask/airway device. FIG. 28 shows various contemplated positions for a pressure relief valve in components associated with a key assembly, according to some embodiments. In the illustrated embodiment, key assembly 2800 (which may be, for example, any one of key assemblies 1200, 1300, 1400, 1500) is connected to airway device/mask 170 by tubing 2810 and connectors 2820A, 2820B.

In various embodiments, pressure relief valve 2830 (represented by a rectangle with a diagonal hatching pattern) may be positioned in any one of positions A, B, C, or D. For example, as shown in FIG. 28, in one embodiment, pressure relief valve 2830 is placed in position A in tubing 2810. In another embodiment, pressure relief valve 2830 is placed in position B in connector 2820A. In yet another embodiment, pressure relief valve 2830 is placed in position C in connector 2820B. In yet one more embodiment, pressure relief valve 2830 is placed in position D in airway device/mask 170. Some embodiments may also be contemplated where pressure relief valve 2830 is placed in the key assembly itself. For instance, as shown in FIG. 28, pressure relief valve 2830 may be placed in position E, which is in air tube 2802 of key assembly 2800. As described herein, air tube 2802 may be connected to system outlet 112 on device 100.

In any of the illustrated embodiments, pressure relief valve 2830 provides pressure relief in association with key assembly 2800 and the specified ventilation parameters for rescue breath determined by a key of the key assembly. Thus, pressure relieve valve 2830 may be specifically designed to operate according to the specified ventilation parameters associated with key assembly 2800. Further, as the different key assemblies described herein may have different flow rates or operating pressures, pressure relief valves 2830 may be individually selected for the different key assemblies to provide pressure release at the desired pressure.

In some embodiments, a PEEP (positive end expiratory pressure) valve may be positioned in any one of positions A, B, C, or D. The PEEP may valve may be implemented instead of pressure relief valve 2830 or in addition to the pressure relief valve. The PEEP valve may be implemented to add a small amount of pressure at the end of a breath. Adding the small amount of pressure at the end of the breath may improve oxygenation of the patient.

Embodiments with Compression Pad

In many instances, it is recommended that pulsed rescue breaths not be provided by ventilators (such as device 100) during chest compressions when airway devices are used on the patient. As described herein, device 100 is operated pneumatically and thus electrical processes for preventing rescue breaths during compression are not implementable without the addition of electrical power. The addition of electrical power to device 100, however, is not desired for the various safety reasons described herein. To overcome the issues to prevent pulsed breaths during compression, the present disclosure contemplates a compression pad that detects chest compressions being connected to airway devices that are used with device 100.

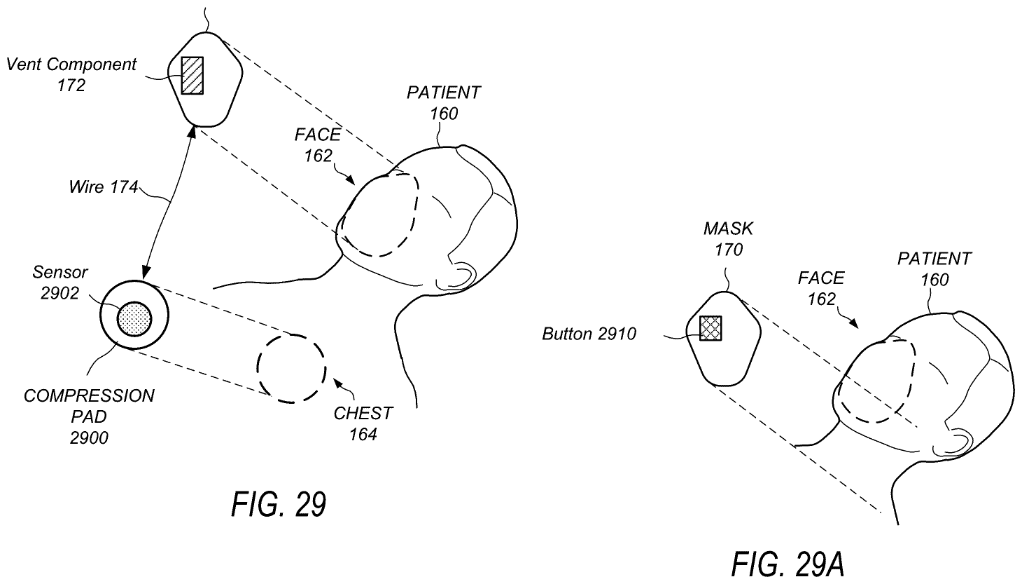

FIG. 29 depicts an example representation of implementation of a compression pad with an airway device in treatment of patient 160, according to some embodiments. In the illustrated embodiment, compression pad 2900 is connected to airway device/mask 170. In certain embodiments, compression pad 2900 is only connected to airway devices that are masks. Constant air flow devices or other airway devices that do not need to have breaths inhibited during compression may not have a compression pad attached to the device. In various embodiments, compression pad 2900 is provided as part of a kit along with airway device 170. The kit may include, for example, a package with the compression pad and one or more of disposable corrugated tubing, a connector, and, an airway device. In certain embodiments, compression pad 2900 is already connected to airway device 170 to ensure both components are used together.

Compression pad 2900, as shown in FIG. 29, may be positioned on chest 164 of patient 160 when airway device 170 is used on the patient. Compression pad 2900 may monitor chest compressions (either automatic or manual) of the patient. Compression pad 2900 may have a size and materials selected to be comfortably placed on chest 164 of patient 160. For instance, in one embodiment, compression pad 2900 has a diameter of three inches and thickness of a quarter of an inch, and the pad will be made out of platinum cure silicone rubber compound.

In some embodiments, compression pad 2900 may include compression sensor 2902 inside the pad. In one embodiment, sensor 2902 is a square FSR sensor. When more pressure is applied to the sensing (square) area of sensor 2902 (e.g., the FSR sensor), the resistance is lowered. Sensor 2902 detects any force applied on any part of the square surface area of the sensor. In certain embodiments, sensor 2902 is used to detect when compressions are made over compression pad 2900 during CPR based on a predetermined pressure. For instance, when the pressure applied is above the predetermined pressure, a compression is detected whereas when the pressure is below the predetermined pressure, no compression is detected. The compression pad 2900 may have a relatively small size such that the pad can be used on different sized patients (e.g., adults, children, and infants). The different sized pads may be connected to different sized airway devices according to the intended patient size.

With compression pad 2900 connected to airway device 170, compressions detected by the compression pad may be monitored to determine operation of the airway device. For instance, in certain embodiments, airway device 170 includes vent component 172. Vent component 172 may redirect breaths provided by device 100 to be vented to the atmosphere instead of being provided to the patient when compressions are detected by compression pad 2900. Vent components 172 may alternatively be positioned in a connector or tubing coupled to airway device 170 (e.g., tubing 2810 or connector 2820, shown in FIG. 28). Connection between vent component 172 and compression pad 2900 may be, for example, an electrical or non-electrical connection where compressions detected by the compression pad trigger opening of the vent component to the atmosphere. Thus, when compressions are detected, vent component 172 vents breaths from device 100 to the atmosphere and inhibits breaths being provided to the patient, according to the generally accepted protocol for CPR. In some embodiments, a controller (e.g., any controller described herein) may be coupled to vent component 172 and compression pad 2900. The controller may receive indications of compressions from compression pad 2900 and control venting by vent component 172.

In various embodiments, as described above, compression pad 2900 is provided in a kit or package along with airway device 170 (and its associated key assembly). Thus, when the kit or package is opened to treat the patient, both airway device 170 and compression pad 2900 are placed on patient 160. Kits utilizing advanced airway devices (e.g., an endotracheal tube) or other advanced airway devices may not have a compression pad to simplify the system and to prevent the possibility of blocking breaths when continuous ventilation is desired. In certain embodiments, compression pad 2900 and vent component 172 are connected by wire 174 though additional embodiments with wireless connections between the components may be contemplated.

In some embodiments, a button for pausing breaths is placed on mask 170 (or a component connected to the mask). FIG. 29A depicts an example representation of implementation of a breath pause button with an airway device in treatment of patient 160, according to some embodiments. In the illustrated embodiment, button 2910 is located on the outside of mask 170 though the button may be located elsewhere on the ARBU device. Button 2910 may be activated (e.g., pressed) to provide breath interruption to patient 160 by pausing/stopping the flow of air being provided to the patient. Operation of button 2910 may, for example, activate a device inside mask 170 (or the airway connected to the mask) to block the flow of air while the button is depressed. Release of button 2910 may stop the block of air flow. In some embodiments, button 2910 may activate venting of breaths from device 100 to the atmosphere to inhibit breaths being provided to the patient.

In various embodiments when button 2910 and airflow to mask 170 is blocked, pressure may build in the ARBU device, for instance, if the on-delay timer and reset modules have started a breath pulse. A pressure relief valve, as described herein, may vent the pressure when the pressure builds beyond the predetermined relief pressure of the valve to prevent excess pressure build-up in the ARBU device. In some embodiments, if button 2910 is released at the end of a breath cycle and the breath has already passed through the pressure relief valve, the next breath cycle may be delayed (e.g., for about 5 seconds). If the breath blocked by button 2910 occurs prior to the breath pulse set by the on-delay timer and reset modules, the on-delay timer may reset and then, when the button is released, the next breath may be delivered. In some embodiments, pressing of button 2910 may reset the on-delay timer. Resetting of the on-delay timer may allow the next breath pulse to be provided immediately after button 2910 is released to avoid starting the breath pulse in the middle of a breath profile.

### Multiple ARBU Device Embodiments

As described herein, various embodiments of device 100 include a single device that provides three different pulsed rescue breaths for different types of patients (e.g., adult, child, infant) in addition to providing a constant flow rate option. To accomplish this, device 100 may utilize four different air channels that are connected to a single inlet and a single outlet. The present disclosure also contemplates various embodiments that separate the different air channels into individual devices. Separation of the air channels into individual devices may, for example, provide the possibility of implementing the devices as disposable devices (e.g., single use devices) that are intended for a specific type of patient, though the devices may also be reusable for any number of uses. The disposable devices may be of a small size (e.g., about the size of a bar of soap) to allow easy storage and use.

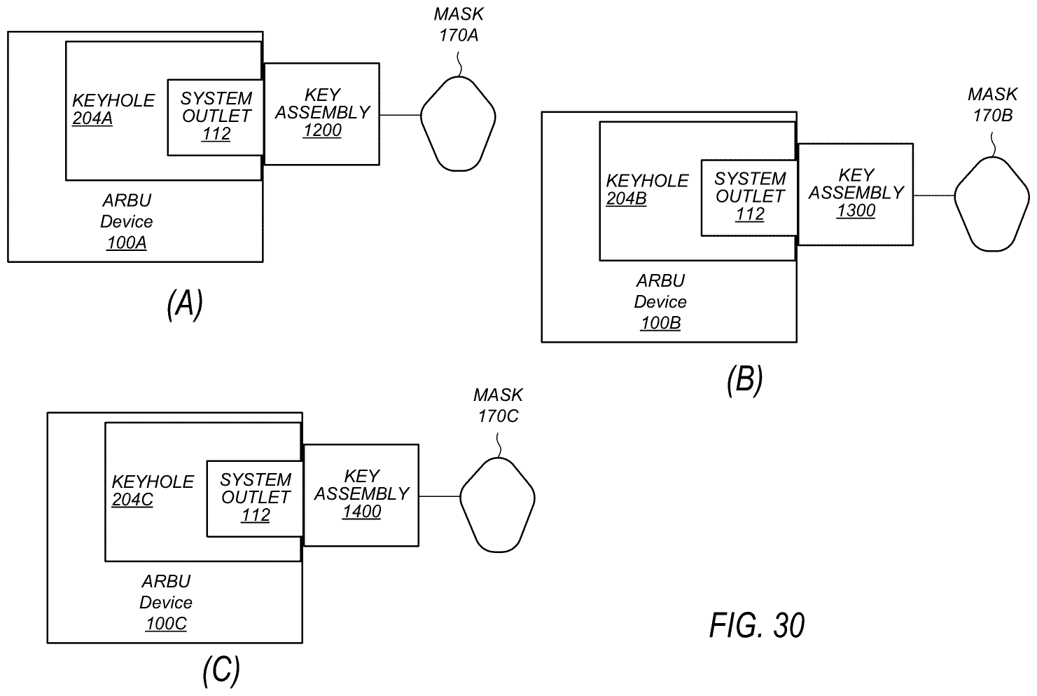

FIG. 30 depicts a block diagram overview of three separate ARBU devices intended for different types of patients, according to some embodiments. In the illustrated embodiment, there are three ARBU devices 100A, 100B, 100C. ARBU device 100A may be intended for adult patients, ARBU device 100B may be intended for child patients, and ARBU device 100C may be intended for child patients. Accordingly, airway device 170A is sized for adult patients, airway device 170B is sized for child patients, and airway device 170C is sized for infant patients. ARBU devices 100A-C may be sized and designed for use in the different types of patients according to the specifications described herein.

In certain embodiments, each ARBU device 100A-C includes a keyhole corresponding to the key assembly associated with the airway device of the specific patient type intended for use with the ARBU device. For example, as shown in FIG. 30, ARBU device 100A includes keyhole 204A that corresponds to key assembly 1200 and airway device 170A, which are all intended for adult patients. Similarly, ARBU device 100B includes keyhole 204B that corresponds to key assembly 1300 and airway device 170B, which are all intended for child patients, and ARBU device 100C includes keyhole 204C that corresponds to key assembly 1400 and airway device 170C, which are all intended for infant patients.

In some embodiments (e.g., for use by emergency personnel), ARBU devices 100A-C may have their corresponding key assemblies and airway devices permanently attached such that the personnel simply grabs the entire unit as a whole or the key assemblies and the airway devices may be unattached and connected at the point of use. Attachment of ARBU devices 100A-C to airway devices 170A-C may be, for example, a direct connection or via a connecting tube (such as corrugated anesthesia tubing). In some embodiments (e.g., for more advanced personnel), ARBU devices 100A-C may be selected separately from airway devices 170A-C and the airway devices are attached to the ARBU devices at the time of treatment. Various measures may be taken to inhibit connection of a wrong airway device to an ARBU device (e.g., connecting a child airway device to an adult ARBU device. For instance, color-coding or different diameters connections, as described below, may be used to inhibit wrongly sized airway devices being connected to specific ARBU devices.

Each ARBU device 100A-C provides the specific rescue breath profile (as described herein) based on the intended patient. For example, ARBU device 100A provides an adult patient rescue breath profile, ARBU device 100B provides a child patient rescue breath profile, and ARBU device 100C provides an infant rescue breath profile. Thus, in the case of an emergency involving a patient, the ARBU device corresponding to the type of patient (e.g., adult, child, or infant) may be selected and utilized on the patient. The selected ARBU device may then provide the rescue breath profile that corresponds to the patient. In some embodiments, the rescue breath profile is provided automatically (e.g., according to the internal pneumatics of the selected ARBU device).

In certain embodiments, the rescue breath is provided without any deviation allowed from the predetermined rescue breath profile. In some embodiments, a user adjustable range may be provided in one or more breath parameters of the ARBU device. For example, a user adjustable range may be provided for adjusting respiratory rate and/or breath volumes within a certain range. While there may be a user adjustable range, the range may be limited to only be adjustable within safe limits for the type of patient for which the ARBU device is intended. For instance, an ARBU device intended for a child patient may be adjustable only within respiratory rate and/or breath volumes that are safe for child patients. Allowing varying breath parameters within safe limits provides adjustability for advanced practitioners while maintaining the safety of the ARBU device and reducing the potential for harm to the patient.

Some embodiments may be contemplated for further division of ARBU devices into more categories than just adult, child, and infant. For example, there may be multiple sized ARBU devices within the child category as children may be of various sizes. Further subdivision of the child category may be based on age, height, and/or weight of a child. Any number of sub-categories may be provided. Also, sub-categories could be applied to adults as well (e.g., small, large, x-large, etc. based on height and/or weight). The ARBU device specific for a sub-category may have a specific size mask or airway device (e.g., LMA (laryngeal mask airway) device). Additionally, the sub-categories may have specific breath parameters (e.g., breath rates and/or volumes) assigned to them. Thus, there may be many different sizes ARBU devices available beyond the 3 main categories of adult, child, and infant to provide further specialization and allow faster response times with improved safety during emergency situations.

TABLE II presents examples of possible color-coded sub-categories for children that may be based on weight and age for use with ARBU devices.

TABLE II

| | | |
|---|---|---|
| Color | Weight Range | Age Range |

| | | | | |
|---|---|---|---|---|
| Pink | 5-7 | kg | 3-5 | months |
| Red | 8-9 | kg | 6-11 | months |
| Purple | 10-11 | kg | 12-24 | months |
| Yellow | 12-14 | kg | 2 | years |
| White | 15-18 | kg | 3-4 | years |
| Blue | 19-23 | kg | 5-6 | years |
| Orange | 24-29 | kg | 7-8 | years |
| Green | 30-36 | kg | 9-10 | years |

| | | | |
|---|---|---|---|
| Grey | 3-5 | kg | 0-3 months |
| | | | (neonates) |

The multiple categories and sub-categories of ARBU devices may have identification systems for faster recognition and deployment. For instance, the ARBU devices may be color-coded or otherwise marked for the specific category or sub-category according to the colors in TABLE II. As an example, in a hospital setting, a child may be given a color-coded bracelet where the bracelet is color-coded based on the age, height, and weight of the child according to the charts in TABLE II. The color-coded bracelet corresponds to emergency resuscitation protocols for drug dosages, breathing tubes/airway device sizes, breathing profiles, etc. for the child. Information may be kept in a color-coded cart. The cart may, for instance, have color-coded drawers corresponding to the color-coded bracelets with all the various supplies for a particular color located in that color drawer. Thus, in an emergency situation, the color-coded drawer corresponding to the patient's bracelet can be opened and the correct supplies are easily acquired.

In certain embodiments, an ARBU device specific to the age, height, and/or weight of the patient is obtained by a practitioner. For example, the ARBU device may be in the color-coded drawer chosen based on the color-coded bracelet worn by the patient. Because of the color-coding, the ARBU device may be pre-set to provide proper ventilation settings for the patient with that color-coded bracelet. In various embodiments, the proper airway device for the patient (e.g., mask or LMA) may be located in the same drawer. In some embodiments, a particular ARBU device may only be used with particular airway devices (e.g., particular endotracheal tubes, masks, or LMAs sized for a particular category/sub-category of patient). For instance, a 6-year old patient in the purple category may receive an ARBU device that only fits with a 3.5 cuffed endotracheal tube or a number 2 LMA mask). In some embodiments, the airway devices may be color-coded in the same manner as the ARBU devices and the drawers. In various instances, airway devices of the same size may have multiple colors as they may be used with multiple categories of patients. For example, purple, yellow, white, and blue may all correspond to a number 2 LMA mask. In some embodiments, different ARBU devices and airway devices may have different diameter attachments (e.g., connectors or hoses) that allows only a specific airway device to be connected to a specific ARBU device (e.g., an adult airway device can only be attached to an adult ARBU device).

Additionally, the ARBU device may have initial settings that correspond to the color-coding. The initial settings may be, for example, a median value within a range for a particular color-coded category patient. TABLE III shows examples of ranges for color-coded ARBU devices according to the color-coded ranges in TABLE II.

TABLE III

| | Tidal Volume | Inspiration |
|---|---|---|
| Color | (mL) | Time (sec) |
| Pink | 40-65 | 0.6 |
| Red | 50-85 | 0.6 |
| Purple | 65-105 | 0.7 |
| Yellow | 80-130 | 0.7 |
| White | 100-165 | 0.7 |
| Blue | 125-210 | 0.8 |
| Orange | 160-265 | 0.8 |
| Green | 200-330 | 0.8 |

A practitioner may be allowed to vary the settings of the ARBU device within these ranges from the initial settings of the ARBU device. Thus, the practitioner may be able to adjust/compensate for changes in the patient's condition within ranges considered safe for the particular patient. For example, the practitioner may want to adjust the initial settings based on the clinical state of a patient and/or arterial blood gas monitoring, or other factors, to provide more directed care to the patient's immediate needs.

The order of the blocks of the methods may be changed, and various elements may be added, reordered, combined, omitted, modified, etc. Various modifications and changes may be made as would be obvious to a person skilled in the art having the benefit of this disclosure. The various embodiments described herein are meant to be illustrative and not limiting. Many variations, modifications, additions, and improvements are possible. Accordingly, plural instances may be provided for components described herein as a single instance. Boundaries between various components, operations and data stores are somewhat arbitrary, and particular operations are illustrated in the context of specific illustrative configurations. Other allocations of functionality are envisioned and may fall within the scope of claims that follow. Finally, structures and functionality presented as discrete components in the example configurations may be implemented as a combined structure or component. These and other variations, modifications, additions, and improvements may fall within the scope of embodiments as defined in the claims that follow.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed or omitted, and certain features of the invention may be utilized independently, all as would be apparent to one skilled in the art after having the benefit of this description of the invention. Changes may be made in the elements described herein without departing from the spirit and scope of the invention as described in the following claims. The words “include”, “including”, and “includes” mean including, but not limited to. As used throughout this application, the singular forms “a”, “an” and “the” include plural referents unless the content clearly indicates otherwise. Thus, for example, reference to “a mask” includes a combination of two or more masks. The term “coupled” means directly or indirectly connected.

What is claimed is:

1. A rescue breath apparatus, comprising:
a ventilator device configured to be coupled to an air/oxygen source, the ventilator device comprising:
an air channel configured to provide a rate and volume of rescue breaths specified for a patient; and
a system outlet coupled to the air channel, the system outlet being configured to output air/oxygen from the device;
an airway device configured to be placed on a face of the patient;
an air tube configured to be coupled between the system outlet and the airway device;
a compression pad configured to be positioned on a chest of the patient, the compression pad having a sensor configured to detect chest compressions being performed on the patient; and
a vent component attached to the airway device and coupled to the compression pad, wherein the vent component is configured to vent breaths from the ventilator device to atmosphere when the compression pad detects chest compressions being performed on the patient.

2. The apparatus of claim 1, wherein the vent component communicates with the compression pad, and wherein detection of compressions by the compression pad triggers the vent component to vent breaths to the atmosphere.

3. The apparatus of claim 1, wherein, when the vent component vents breaths to the atmosphere, no breaths are provided to the patient.

4. The apparatus of claim 1, further comprising a controller coupled to the vent component and the compression pad, wherein the controller is configured to control venting by the vent component based on receiving indications of compressions being performed by the compression pad.

5. The apparatus of claim 1, wherein the ventilator device includes:
a keyhole assembly comprising a valve controlling air flow through the air channel; and
a key assembly attached to the airway device, wherein the key assembly includes an air tube configured to be coupled to the system outlet and a key configured to engage the valve in the keyhole assembly when the air tube is coupled to the system outlet.

6. The apparatus of claim 5, wherein engagement of the key with the valve in the keyhole assembly opens air/oxygen flow through the air channel corresponding to the keyhole assembly.

7. The apparatus of claim 5, wherein the key engages the valve in the keyhole assembly by pushing a button on the valve to open the valve.

8. The apparatus of claim 5, wherein the keyhole assembly includes a keyhole for receiving the key from the key assembly.

9. A rescue breath apparatus, comprising:
a ventilator device configured to be coupled to an air/oxygen source, the ventilator device comprising:
an air channel configured to provide a rate and volume of rescue breaths specified for a patient; and
a system outlet coupled to the air channel, the system outlet being configured to output air/oxygen from the device;
an airway device configured to be placed on a face of the patient;
an air tube configured to be coupled between the system outlet and the airway device; and
a breath pause button, wherein the breath pause button is configured to be activated to inhibit breaths from being provided to the patient while the button is activated.

10. The apparatus of claim 9, further comprising:
a compression pad configured to be positioned on a chest of the patient, the compression pad having a sensor configured to detect chest compressions being performed on the patient; and
a vent component attached to the airway device and coupled to the compression pad, wherein the vent component is configured to vent breaths from the ventilator device to the atmosphere when the compression pad detects chest compressions being performed on the patient.

11. The apparatus of claim 9, wherein the ventilator device includes:
a keyhole assembly comprising a valve controlling air flow through the air channel; and
a key assembly attached to the airway device, wherein the key assembly includes an air tube configured to be coupled to the system outlet and a key configured to engage the valve in the keyhole assembly when the air tube is coupled to the system outlet.

12. A rescue breath kit, comprising:
a first ventilator device configured to be coupled to an air/oxygen source, the first ventilator device comprising:
a first air channel configured to provide a rate and volume of rescue breaths specified for an adult patient;
a first system outlet coupled to the first air channel, the first system outlet being configured to output air/oxygen from the first ventilator device;
a second ventilator device configured to be coupled to the air/oxygen source, the second ventilator device comprising:
a second air channel configured to provide a rate and volume of rescue breaths specified for a child patient;
a second system outlet coupled to the second air channel, the second system outlet being configured to output air/oxygen from the second ventilator device;
a third ventilator device configured to be coupled to the air/oxygen source, the third ventilator device comprising:
a third air channel configured to provide a rate and volume of rescue breaths specified for an infant patient;
a third system outlet coupled to the third air channel, the third system outlet being configured to output air/oxygen from the third ventilator device;
a first airway device configured to be placed on a face of the adult patient;
a second airway device configured to be placed on a face of the child patient;
a third airway device configured to be placed on a face of the infant patient;
a first air tube configured to be coupled between the first system outlet and the first airway device;
a second air tube configured to be coupled between the second system outlet and the second airway device; and
a third air tube configured to be coupled between the third system outlet and the third airway device.

13. The rescue breath kit of claim 12, wherein the first airway device is attached to the first system outlet by the first air tube, wherein the second airway device is attached to the second system outlet by the second air tube, and wherein the third airway device is attached to the third system outlet by the third air tube.

14. The rescue breath kit of claim 12, wherein the first airway device and the first air tube are only usable with the first ventilator device, the second airway device and the second air tube are only usable with the second ventilator device, and the third airway device and the third air tube are only usable with the third ventilator device.

15. The rescue breath kit of claim 12, wherein the first airway device, the first air tube, and the first ventilator device only provides the rate and volume of rescue breaths specified for the adult patient.

16. The rescue breath kit of claim 12, wherein the second airway device, the second air tube, and the second ventilator device only provides the rate and volume of rescue breaths specified for the child patient.

17. The rescue breath kit of claim 12, wherein the third airway device, the third air tube, and the third ventilator device only provides the rate and volume of rescue breaths specified for the infant patient.

18. The rescue breath kit of claim 12, wherein the airway devices, the air tubes, and the ventilator devices are for a single use on a specified patient.

19. The rescue breath kit of claim 12, further comprising:
a fourth ventilator device configured to be coupled to the air/oxygen source, the fourth ventilator device comprising:
a fourth air channel configured to provide a rate and volume of rescue breaths specified for a second child patient, the second child patient having a different age and/or weight from the child patient for the second air channel;
a fourth system outlet coupled to the fourth air channel, the fourth system outlet being configured to output air/oxygen from the fourth ventilator device;
a fourth airway device configured to be placed on a face of the second child patient; and
a fourth air tube configured to be coupled between the fourth system outlet and the fourth airway device;
wherein the second ventilator device and the fourth ventilator device are color-coded with different colors, a color of the second ventilator device corresponding to an age and/or weight of the child patient and a color of the fourth ventilator device corresponding to an age and/or weight of the second child patient.

20. The rescue breath kit of claim 12, further comprising:
a compression pad coupled to at least one airway device of the first, second, and third airway devices, the compression pad being configured to be positioned on a chest of the patient, the compression pad having a sensor configured to detect chest compressions being performed on the patient; and
a vent component attached to the at least one airway device and coupled to the compression pad, wherein the vent component is configured to vent breaths from the ventilator device associated with the at least one airway device to the atmosphere when the compression pad detects chest compressions being performed on the patient.
